(19) United States
(12) Patent Application Publication
(10) Pub. No.: US 2008/0041403 A1
(43) Pub. Date: Feb. 21, 2008

(54) Treatment of internal rotation deformity in children caused by an injury to the brachial plexus

(76) Inventor: Rahul K. Nath, Houston, TX (US)

Correspondence Address: THE MATTHEWS FIRM, 2000 BERING DRIVE, SUITE 700, HOUSTON, TX, 77057, US

(21) Appl. No.: 11/726,314

(22) Filed: Mar. 20, 2007

Related U.S. Application Data
(60) Provisional application No. 60/784,170, filed on Mar. 21, 2006.

(51) Int. Cl. A61B 19/00

(52) U.S. Cl. 128/898

(57) ABSTRACT

Following a diagnosis of a Brachial Plexus injury to a child involving the elevation and rotation of the scapula, surgical intervention is used to reposition the clavicle and the acromion, which are then allowed to re-align and interlock with the scapula, thus tilting the triangle containing the clavicle, acromion and scapula.

### RELATED APPLICATION

This application claims priority under U.S. Provisional Application No. U.S. 60/784,170 filed on Mar. 21, 2006.

### BACKGROUND OF THE INVENTION

The brachial plexus is an arrangement of nerve fibers (a plexus) running from the spine (vertebrae C5-T1), through the neck, the axilla (armpit region), and into the arm. The brachial plexus is responsible for cutaneous and muscular innervation of the entire upper limb, with two exceptions: the trapezius muscle innervated by the spinal accessory nerve and an area of skin near the axilla innervated by the intercostobrachialis nerve.

The five roots are the five anterior rami of the spinal nerves, after they have given off their segmental supply to the muscles of the neck.

These roots merge to form three trunks:
“superior” or “upper” (C5-C6)
“middle” (C7)
“inferior” or “lower” (C8-T1)

Each trunk then splits in two, to form six divisions:
anterior division of the superior, middle, and inferior trunks
posterior division of the superior, middle, and inferior trunks

These six divisions will regroup to become the three cords. The cords are named by their position in respect to the axillary artery.
The posterior cord is formed from the three posterior divisions of the trunks (C5-T1)
The lateral cord is the anterior divisions from the upper and middle trunks (C5-C7)
The medial cord is simply a continuation of the lower trunk (C8-T1)

Brachial Plexus injury, or Erbs Palsy, can be severe and permanent. This has been generally handled through management protocols, such as physical therapy and electrical stimulation.

Generally, if there is continued significant shoulder, elbow or hand weakness at 4 to 6 months after injury, brachial plexus surgical exploration is sometimes indicated.

The most commonly injured elements of the brachial plexus in children are the upper roots. These injuries often lead to loss of abduction and external rotation of the shoulder due to deltoid, supraspinatus, infraspinatus and teres minor denervation. This results in a muscle imbalance that eventually causes contractures to develop about the shoulder joint. Surgery has been used in the treatment of these contractures with great success in the restoration of shoulder abduction. However, the internal rotation posture in certain cases may have an additional component of bony rotation. This has significant functional consequences including limb shortening, loss of supination, winging of the scapula and lateral angulation of the elbow.

Traditionally, the approach to this problem has been the use of derotational humeral osteotomy, i.e., the treatment involving merely the manipulation of the involved bones and the spine to neutralize the hand and forearm. This approach attempts to restore the arm to a neutral position and often does do so. It does not address the primary cause of internal rotation, so further management may be necessary.

Based on 3D Cat scans, it is apparent that the internal rotation of the arm associated with obstetrical brachial plexus injury is often related to a change in the spatial relationship of the scapula and clavicle. The elevation and forward rotation of the scapula causes positional changes in the acromio-clavicular (AC) joint. The bony changes are collectively known as a SHEAR deformity (Scapular Hypoplasia, Elevation and Rotation). There are 5 grades (0 through 4). As a result, internal rotation posture of the arm is inevitable with attendant functional and visible consequences. Physical therapy cannot significantly improve the appearance or function of the severely elevated scapula.

### BRIEF DESCRIPTION OF THE DRAWING

FIG. 1, the sole drawing FIGURE, graphically illustrates the triangle formed by the clavicle, the scapula and the acromion, which is tilted in accord with the present invention.

### DETAILED DESCRIPTION OF THE PREFERRED EMBODIMENT OF THE INVENTION

In human anatomy, the clavicle or collar bone is classified as a long bone that makes up part of the shoulder girdle (pectoral girdle). It receives its name from the Latin clavicula (“little key”) because the bone rotates along its axis like a key when the shoulder is abducted. (This movement is palpable with the opposite hand). In some people, particularly females who may have less fat in this region, the location of the bone is clearly visible as it creates a bulge in the skin.

The clavicle is a doubly-curved long bone (the only horizontal long bone in the human body) that connects the arm (upper limb) to the body (trunk), located directly above the first rib. Medially, it articulates with the manubrium of the sternum (breast-bone) at the sternoclavicular joint. At its lateral end it articulates with the acromion of the scapula (shoulder blade) at the acromioclavicular joint. It is shorter, thinner, less curved, and smoother in women than in men. It has a rounded medial end and a flattened lateral end.

In anatomy, the scapula, or shoulder blade, is the bone that connects the humerus (arm bone) with the clavicle (collar bone).

The scapula forms the posterior part of the shoulder girdle. In humans, it is a flat bone, roughly triangular in shape.

It has two surfaces, three borders, and three angles. The anterior (front) side of the scapula shows the fossa subscapularis (subscapular fossa) to which the subscapularis muscle attaches.

The posterior surface of the scapula is divided by a bony projection, the spina scapulae (opposite to the fossa subscapularis) into the supraspinous fossa and the infraspinous fossa. This projection is called the spine of the scapula. It begins flat at the base of the shoulder bone, ascends in distal direction for all animals but carnivores and humans to its peak at about the middle of the scapula, this peak is called tuber scapulae. After this peak the spina scapulae steeply decays in height. For humans and carnivores and bovinae the spina runs into a forward pointing hook called acromion, which continues past the main part of the bone.

Another hook-like projection comes off the lateral angle of the scapula, and is called the coracoid process. The end of this hook is the site of attachment of many muscles, such as the coracobrachialis muscle.

Near the base of the coracoid process, so also on the lateral angle, there is a depression called the glenoid cavity. This forms the socket that the head of the humerus articulates with.

The scapula also articulates with the clavicle, via the acromion process (the acromioclavicular joint).

The acromion process, or simply the acromion, is an anatomical feature on the scapula. It is a continuation of the scapular spine, and hooks over anteriorly.

The acromion articulates with the clavicle to form the acromioclavicular joint.

The acromion forms the summit of the shoulder, and is a large, somewhat triangular or oblong process, flattened from behind forward, projecting at first lateralward, and then curving forward and upward, so as to overhang the glenoid cavity.

Surfaces

Its superior surface, directed upward, backward and lateralward, is convex, rough, and gives attachment to some fibers of the Deltoideus, and in the rest of its extent is subcutaneous.

Its inferior surface is smooth and concave.

Borders

Its lateral border is thick and irregular, and presents three or four tubercles for the tendinous origins of the Deltoideus.

Its medical border, shorter than the lateral, is concave, gives attachment to a portion of the Trapezius, and presents about its center a small, oval surface for articulation with the acromial end of the clavicle.

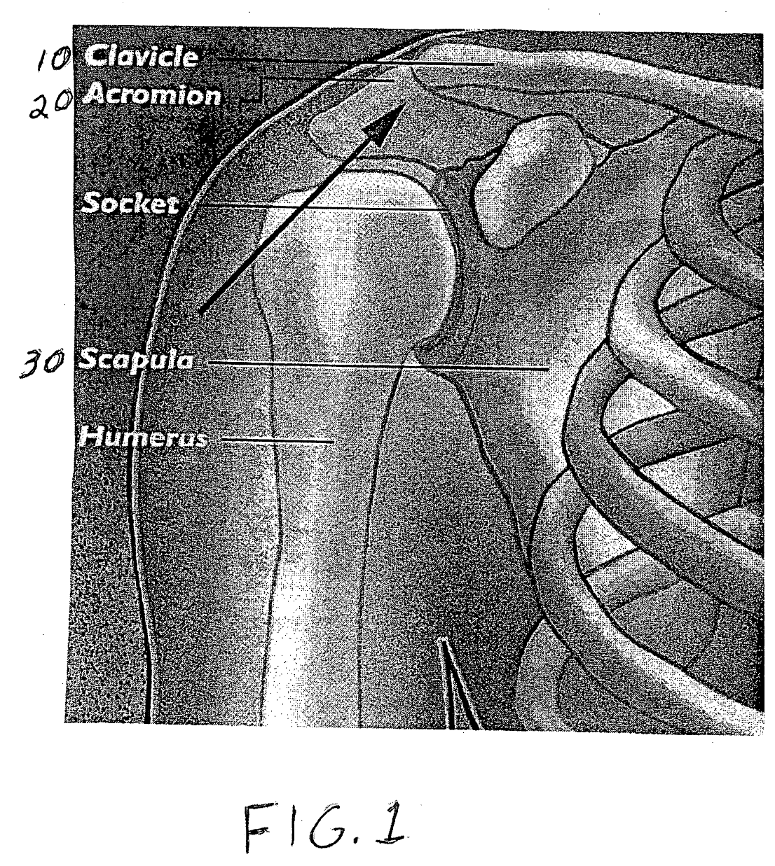

Referring now to the drawing in more detail, FIG. 1 graphically illustrates the “triangle” formed by the clavicle 10, the acromion 20 and the scapula 30.

The surgical procedure of this invention, combined with capsulodesis, restores the arm to a more natural position by addressing the primary issue of scapular elevation. The clavicle 10 and acromion 20 are repositioned in a controlled fashion and allowed to re-align and interlock naturally, effectively restoring the spatial relationship of the clavicle 10, the acromion 20 and the scapula 30 (tilting the triangle). A posterior gleno-humeral capsulodesis is also performed at this time, to tighten the laxity of the shoulder that has been created by the malpositioned humeral head.

A “gunslinger” splint (arm at the side with elbow bent at 90 degrees) is worn for a period of six weeks following surgery to allow for the newly tilted triangle to heal and strengthen. A direct consequence of this intervention is correction of the internal rotation deformity and funcational improvements in supination and hand-to-mouth movements. Additionally, the length of the arm is significantly improved, because of the repositioning of the gleno-humeral joint and better elbow extension. Therefore, this surgery is an effective improvement on traditional surgical methods.

While there are other surgical procedures know in this art designed to address internal rotation in children with Obstetrical Brachial Plexus Injury (OBPI), the present invention is unique in that it identifies scapular elevation as being the cause of internal rotation deformity, as contracted with merely being a symptom of OBPI.

### EXAMPLE OF SURGERY ACCORDING TO THE INVENTION

In recognizing the scapular elevation as being the cause of internal rotation deformity, the following surgery example is presented.

Preoperative Diagnosis: Right brachial plexus injury with secondary and tertiary deformities.

Post Operative Diagnosis: Abnormal elevation and forward tilt of acromion process and clavicle with posterior subluxation of glenohumeral joint.

TITLES OF PROCEDURES:

1. Osteotomy of right clavicle.

2. Osteotomy of right acromion process.

3. Posterior capsulodesis of right glenohumeral joint.

4. Onlay of right distal clavicle with approximation superiorly to proximal clavicle.

5. Onlay of distal acromion process inferior to proximal spine of the scapula.

Anesthesia: General endotracheal

Indications for Operation: This child had suffered a severe right brachial plexus injury at the time of birth. This resulted in numerous soft tissue and boy deformities related to the growth disturbance arising from the initial nerve injury. One of the previous soft tissue deformities, contractures in the axilla and chest had been previously released surgically with good outcome and improvement in overhead abduction. However, the patient was left with a persistent internal rotation deformity related by preoperative CT scans to an elevated right scapula with forward rotation. This placed the humorous into an internally rotated position in the glenohumeral joint and therefore, resulted in the abnormal posture prior to surgery. The patient had inability to supinate, and external rotation was awkward, The patient also has a persistent elbow-bent posture and shortening of the arm. These were all reasons to perform the surgery, as scheduled. The patient's parents were counseled extensively prior to the surgery and understood definitively the risks and benefits prior to proceeding. They understood that the surgery was a new procedure, although based on sound anatomic and physiologic planning. They enthusiastically wished to proceed with surgery as scheduled.

Operative Note: The patient was brought to the operating room, where he underwent general anesthesia. The right upper extremity and shoulder were prepped and draped in the usual sterile fashion. The patient was placed in the lawn-chair position. After general anesthetic was administered, an incision was created over the distal clavicle and using a Micro-Hall sagittal saw, an osteotomy was completed. An incision was now created over the distal acromion process and similarly an osteotomy was completed. The triangle of the distal acromion process and the distal clavicle, along with the humeral head, now rotated spontaneously into a more neutral position from its forward tilted position.

This places the arm into a more natural and neutral position. The impingement between the distal triangle as described and the proximal clavicle and scapula was relieved.

Attention was now turned to the posterior shoulder, where an incision was created just at the level of the humeral head. Dissection proceeded down with a muscle splitting dissection through the deltoid to isolate the posterior glenohumeral capsule. The capsule was imbricated in a purse-string fashion with several heavy braided nylon sutures, in order to support the previously lax posterior glenohermeral joint capsule. This assisted the arm in maintaining the neutral rotation position created by the osteotomies of the clavicle and the acromion process.

At this time, the inset of the bones appeared to be quite natural and in the neutral position. The distal clavicle was riding above the proximal clavicle and the distal acromion process was riding below the level of the existing acromion process and spine of the scapula. The arm itself was in a neutral position and the forearm was also neutral.

The wounds were all closed in two layers with absorbable suture and a dry dressing was applied. A previously created split was now placed with the arm in adduction, neutral position of the humorous and eternal rotation as well. The forearm was placed in full supination, and the split was held in place with multiple straps.

The patient tolerated the entire procedure well and was awake and alert and extubated following surgery without incident. There were no complications and blood loss was minimal.

1. In a process for treating an injury to the brachial plexus involving the clavicle, the acromion and an elevated scapula, the method steps comprising:
diagnosing the elevated scapula;
repositioning the clavicle and the acromion; and
allowing the repositioned clavicle and the acromion to re-align and interlock with the scapula.

2. The process according to claim 1, wherein the diagnosing step comprises a CAT scan.

3. The process, according to claim 1, comprising in addition thereto, the osteotomy of the involved clavicle.

4. The process according to claim 1, comprising in addition thereto, the osteotomy of the involved acromion.

5. The process according to claim 1, comprising in addition thereto, the capsulodesis of the involved glenohumeral joint.
